(19) United States
(12) Patent Application Publication
(10) Pub. No.: US 2011/0288114 A1
(43) Pub. Date: Nov. 24, 2011

(54) COMPOSITIONS FOR THE TREATMENT OF FIBROTIC DISEASES OR CONDITIONS

(75) Inventors: Scott Turner, Oakland, CA (US); Mary Wolff, Montana, CA (US)

(73) Assignee: Kinemed, Inc., Emeryville, CA (US)

(21) Appl. No.: 13/001,281

(22) PCT Filed: Jun. 22, 2009
(86) PCT No.: PCT/US09/48185

§ 371 Date: Aug. 15, 2011

Related U.S. Application Data
(60) Provisional application No. 61/074,492, filed on Jun. 20, 2008.

(51) Int. Cl. A61K 31/4355; A61P 25/00; A61P 29/00

(52) U.S. Cl. 514/291

(57) ABSTRACT

A method of treating a fibrotic disease or condition in a patient comprising administering noscapine and a pharmaceutical carrier to said patient.

### CROSS REFERENCE TO RELATED APPLICATIONS

The present invention claims the benefit of priority under 35 U.S.C. §119(e) to U.S. Provisional Application Nos. 61/074,492, filed Jun. 20, 2008, which is incorporated by reference in its entirety.

### BACKGROUND OF THE INVENTION

Fibrosis is a complex disorder that can occur in many different tissues in response to prolonged injury. Fibrosis is generally characterized by abnormal increases in the proliferation of fibroblasts and myofibroblasts, as well as by excessive deposition of collagen and other extracellular matrix (ECM) components. Ultimately, these changes may destroy the normal structure and function of the affected organ, as can occur in liver cirrhosis, pulmonary interstitial fibrosis (IPF), the skin and other organs in systemic sclerosis (scleroderma), transplant rejection, the heart in congestive heart failure, and many other diseases. Conventional treatments involving the use of corticosteroids and immunosuppressant drugs have had little or no effect on reversing or preventing the progression of fibrosis (Wynn, 2007).

Because of the complexity of the underlying pathogenesis, diverse therapeutic interventions have been proposed. It has been suggested that reducing the synthesis, excretion, or polymerization of collagen fibrils might be effective at slowing fibrogenesis. Another line of attack is to enhance collagenase activity in an attempt to break down excess ECM, whereas others have suggested the strategy of neutralizing or opposing those cytokines, such as transforming growth factor beta (“TGF-β”), that stimulate collagen synthesis. Several putative antifibrotic agents have been tested in clinical trials but have failed to demonstrate real efficacy in retarding fibrosis.

Among the factors responsible for the initiation and progression of fibrosis, the recruitment of lymphocytes and fibroblasts/myofibroblasts to the wounded area (Hinz et al, 2007) and the induction of TGF-β (Wells 2000; Verrecchia and Mauviel, 2007) are considered to be critical. A drug that could normalize the movement of fibroblasts and myofibroblasts into wounded tissue, and/or regulate the function of TGF-β would therefore be especially useful in treating fibrosis.

### SUMMARY OF THE INVENTION

Accordingly, in one aspect, the present invention provides a method of treating fibrotic disease or condition in a patient comprising administering noscapine and a pharmaceutical carrier to the patient. In some embodiments the fibrotic disease or condition is delayed; in some embodiments the fibrotic disease or condition is reduced.

In an additional aspect, the invention provides methods of treating fibrotic disease comprising administering a pharmaceutical composition comprising noscapine, including variants thereof, and one, two, three or more anti-fibrotic agents, including but not limited to ACE inhibitors, anti-inflammatory agents, Pirfenidone, Gleevec and Bosentan. The pharmaceutical composition may further comprise a pharmaceutical carrier. Such treatment may result in delayed onset of fibrotic disease symptoms or reduction in severity of fibrotic disease symptoms.

### DESCRIPTION OF THE DRAWINGS

FIG. 1 depicts the synthesis of new collagen (percent of labeled hydroxyproline (OHP) or ‘f’) 2 weeks after a single dose of bleomycin (1.5 U/kg, trans-oral). Noscapine (ip) administration began the same day as the bleomycin and continued for 2 weeks. N=4/group; data are expressed as the mean+SD; * p<0.05 ANOVA followed by Dunnett's test to compare to bleomycin/vehicle.

FIG. 2 depicts the absolute amount of OHP per lung 2 weeks after a single dose of bleomycin (1.5 U/kg; trans-oral) as measured by a chloramine-t assay. Noscapine administration began the same day as the bleomycin and continued for 2 weeks. N=4-5/group; data are expressed as the mean+SD; * p<0.05 one way ANOVA, Dunnett's compared to bleomycin/vehicle control or noscapine-treated (30 mg/kg and 100 mg/kg) groups.

FIG. 3 depicts the absolute amount of newly synthesized collagen (calculated as f×[OHP]) per lung 2 weeks after a single dose of bleomycin (1.5 U/kg; trans-oral). Bleomycin increased the absolute amount of newly synthesized collagen in the lung relative to the vehicle control or noscapine-treated groups. N=4-5/group; data are expressed as the mean+S.D.; * p<0.05 ANOVA followed by Dunnett's test for comparison with bleomycin/vehicle control.

FIG. 4 depicts representative slides and a semi-quantitative analysis of the alpha-smooth muscle actin (α-SMA) positive lung cells obtained from the lungs of mice 2 weeks after a single dose of bleomycin (1.5 U/kg; trans-oral; same animals as in above FIGS. 1-3). Bleomycin significantly increased the number of activated α-SMA containing myofibroblasts present in the lung. Noscapine significantly reduced the number of myofibroblasts activated by bleomycin. N=4-5/group; data are expressed as the mean+S.D.; * p<0.05 ANOVA followed by Dunnett's test for comparison with bleomycin/vehicle control.

FIG. 5 depicts the synthesis of new collagen (percent labeled OHP, or ‘f’) 2 weeks after a single dose of bleomycin (1.5 U, trans-oral). Administration of 300 mg/kg noscapine (po) began the same day as the bleomycin and continued for 2 weeks. N=10/group; data are expressed as the mean+S.D. * p<0.05 1 way ANOVA, Dunnett's compared to bleomycin/vehicle.

FIG. 6 depicts the synthesis of new collagen (percent labeled OHP, or ‘f’) 2 weeks after a single dose of bleomycin (1.5 U, trans-oral). Noscapine was administered in the diet at a concentration that yielded an approximate dose of 300 mg/kg beginning the same day as the bleomycin and continued for 2 weeks. Noscapine tended to reduce the % new OH—P, but the effect was not statistically significant. N=5/group; data are expressed as the mean+S.D.

FIG. 7 depicts the synthesis of new collagen (percent labeled OHP, or ‘f’) in the liver following the administration of ANIT either alone or in combination with noscapine (po). At 30 and 100 mg/kg, noscapine, significantly reduced the ANIT-induced elevation of OHP. N=5/group; data are the mean+SD; * p<0.05 ANOVA followed by Dunnett's test for comparison with ANIT/vehicle multiple comparison.

FIG. 8 depicts the absolute amount of OHP in the liver following the administration of ANIT either alone or in combination with noscapine (po). Noscapine significantly reduced the ANIT-induced elevation of OHP content. N=5/group; data are the mean+SD; * p<0.05 ANOVA followed by Dunnett's test for multiple with ANIT/vehicle control.

FIG. 9 depicts the absolute amount of newly synthesized collagen in the liver (calculated as f×[OHP]) following the administration of ANIT either alone or in combination with noscapine (po), compared to the administration of regular chow. At 30 and 100 mg/kg po, noscapine significantly reduced the ANIT-induced elevation of collagen synthesis. N=5/group; data are the mean+SD; * p<0.05 ANOVA followed by Dunnett's test for comparison with the ANIT/vehicle control.

FIG. 10 depicts representative slides and a semi-quantitative analysis of histologically determined collagen content (Masson's Trichrome stain) from the same animals as in FIGS. 7, 8, and 9. Noscapine significantly decreased ANIT-induced collagen content. Data are the mean+SD; * p<0.05 ANOVA followed by Dunnett's test for comparison with the ANIT/vehicle control.

FIG. 11 depicts the effect of noscapine on ANIT-induced increase in the synthesis of collagen in the liver. Noscapine was administered in the diet at a concentration of 2 g/kg to yield an approximate dose of 300 mg/kg, assuming that a 20 g mouse eats 3 g/day of diet. N=5/group; data are the mean+SD; * p<0.05 ANOVA followed by Dunnett's test for comparison with the ANIT/vehicle control.

FIG. 12 depicts the effect of noscapine on the ANIT-induced increase in the synthesis of collagen in the liver. Noscapine was administered by oral gavage once per day at 300 mg/kg; N=5/group; data are expressed as the mean+SD.

FIG. 13 depicts the structure of noscapine and noscapine analogs.

### DETAILED DESCRIPTION OF THE INVENTION

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel et al., eds., 1987): PCR: The Polymerase Chain Reaction, (Mullis et al., eds., 1994); Current Protocols in Immunology (J. E. Coligan et al., eds., 1991); Short Protocols in Molecular Biology (Wiley and Sons, 1999); and Mass isotopomer distribution analysis at eight years: theoretical, analytic and experimental considerations by Hellerstein and Neese (Am J Physiol 276 (Endocrinol Metab. 39) E1146-E1162, 1999), all of which are incorporated by reference for the needed techniques. Furthermore, procedures employing commercially available assay kits and reagents will typically be used according to manufacturer-defined protocols unless otherwise noted.

Recent in vitro studies have indicated that microtubule (MT) dynamics may play a previously unrecognized role in fibrogenesis. Although MTs are not necessary for motility of all cells, proper functioning of MTs appears to be essential for the directed translocation of large cells such as fibroblasts. Very low concentrations of compounds that inhibit MT dynamics greatly decrease the rate at which large cells can migrate into a wounded area (Liao et al, 1995). This may be an important factor in the recruitment of fibroblasts/myofibroblasts into damaged tissue. Furthermore, MT provide a negative feedback loop in TGF-β/Smad signaling by forming a complex with Smad2, Smad3, and Smad4, thus sequestering the rSmads away from the TGF-β receptor (Dong et al, 2000). The TGF-β signaling cascade and its importance in the induction of fibrosis has been reviewed in many publications (Wells 2000; Verrecchia and Mauviel, 2007).

Noscapine is an orally active drug that binds to tubulin and alters its conformation, thereby altering the rate of the disassembly/re-assembly cycle (i.e., the dynamics) of MT (Ye et al. 1998) without affecting the total polymer mass of tubulin or causing gross MT deformations (Zhou et al. 2002). Originally used as an antitussive agent (Empey et al. 1979), noscapine has recently been reported to be an effective anti-cancer agent with little toxicity to normal tissues or inhibition of immune responses (Ke et al. 2000; Zhou et al 2003). Noscapine has been shown to be beneficial in the treatment of stroke, possibly through its ability to block bradykinin activity (Mahmoudian et al. 2003). Noscapine is a non-narcotic phthalideisoquinoline alkaloid that is derived from opium. However, noscapine lacks analgesic, sedative and respiratory-depressant properties, and does not induce either euphoria or dependence. In clinical trials, noscapine appears to be well tolerated (Mahmoudian et al. 2003). Moreover, in the present disclosure, noscapine has been found to be an effective treatment of fibrotic disease or conditions. As such, the present disclosure provides formulations and methods for the treatment of fibrotic disease or conditions.

The present invention is directed to methods and compositions for the treatment of various fibrotic diseases or conditions. “Treatment” in this context includes delay in onset or severity of symptoms, retardation of mortality, and reduction of symptoms.

In one embodiment, the invention provides methods of treating a fibrotic disease or condition comprising administering to a patient in need of treatment a pharmaceutical composition comprising noscapine or a salt thereof. By “fibrotic disease” is meant a disease or disorder characterized by an increase in fibrous connective tissue in an organ or tissue, such as increases in collagen or other extracellular matrix (ECM) components relative to non afflicted controls. “Fibrotic disease” or conditions include, but are not limited to hepatic cirrhosis, congestive heart failure, fibrotic lung disease, photo-aging, cystic fibrosis of the pancreas and lungs, injection fibrosis, which can occur as a complication of intramuscular injections, endomyocardial fibrosis, idiopathic pulmonary fibrosis of the lung, mediastinal fibrosis, myelofibrosis, retroperitoneal fibrosis, progressive massive fibrosis (a complication of coal workers' pneumoconiosis, nephrogenic systemic fibrosis, scleroderma, kidney fibrosis, fibrosis related to organ transplants, scars, burns and the like (see also a discussion of fibrotic disease or conditions in U.S. Pat. No. 7,449,171, and U.S. patent application Ser. No. 11/064,197, both hereby incorporated by reference in their entirety).

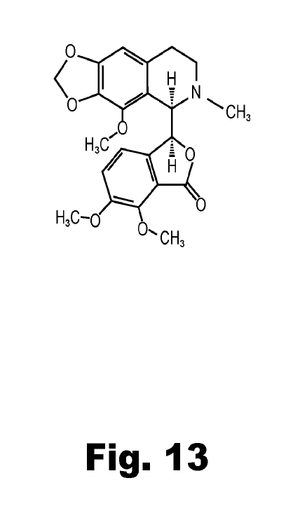

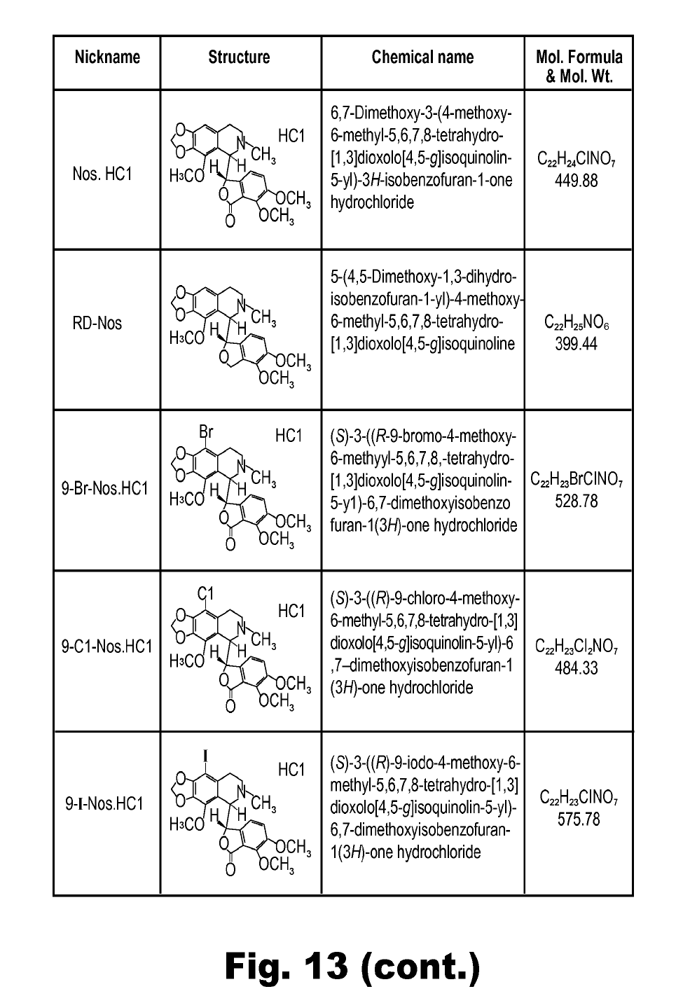

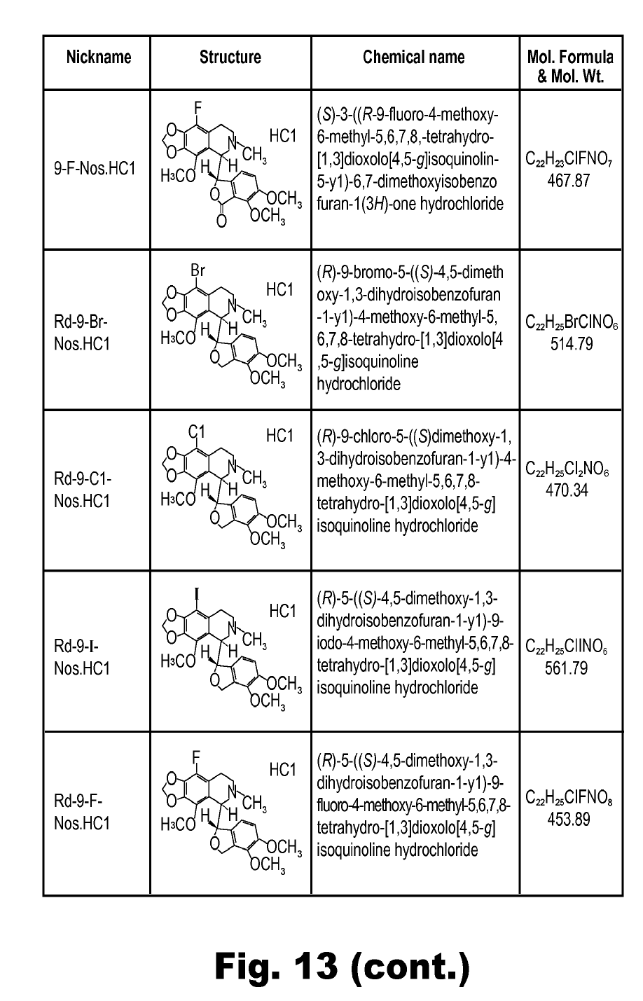

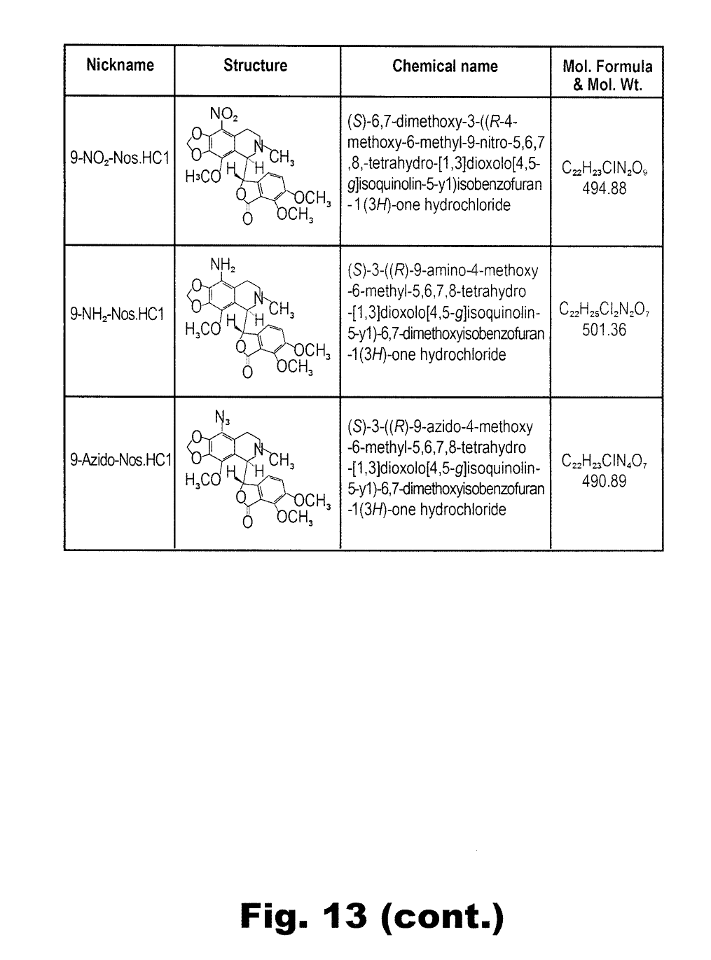

As further described below, one embodiment utilizes noscapine as the agent for treatment. The structure of noscapine is depicted in FIG. 13. “Noscapine” includes noscapine analogs and/or derivatives, for example as are outlined in U.S. Pat. No. 6,376,516, hereby incorporated by reference in its entirety for its specific disclosure related to noscapine analogs and methods of making same. Preferred noscapine analogs are found in FIG. 13.

In another embodiment noscapine is used according to the methods disclosed herein in combination with other agents, preferably other antifibrotic agents. Preferred agents include, but are not limited to Angiotensin Converting Enzyme (ACE) Inhibitors, anti-inflammatory agents, Pirfenidone, Gleevec or Bosentan.

ACE inhibitors can be divided into three groups based on their molecular structure: Sulfhydryl-containing agents, such as Captopril (trade name Capoten), and Zofenopril. Other agents include Dicarboxylate-containing agents. This is the largest group, including: Enalapril (Vasotec/Renitec) Ramipril (Altace/Tritace/Ramace/Ramiwin) Quinapril (Accupril) Perindopril (Coversyl/Aceon) Lisinopril (Lisodur/Lopril/Novatec/Prinivil/Zestril) Benazepril (Lotensin). Finally, other ACE inhibitors include Phosphonate-containing agents, such as Fosinopril (Monopril).

Antiflammatory agents include steroids or non-steroidal anti-inflammatory drugs (NSAIDS). Preferred anti-inflammatory agents include, but are not limited to glucocorticoids, aspirin, ibuprofen, and naproxen.

Formulations

In therapeutic use for the treatment of a fibrotic disease or condition, the compound(s) utilized in the pharmaceutical method of the invention are administered to patients diagnosed with a fibrotic disease or condition or at risk for developing fibrotic disease or conditions, at dosage levels suitable to achieve therapeutic benefit. By “therapeutic benefit” is meant that the administration of compound(s) leads to a beneficial effect in the patient over time.

Initial dosages suitable for administration to humans may be determined from in vitro assays or animal models. For example, an initial dosage may be formulated to achieve a serum concentration that includes the IC₅₀ of the particular metabolically active agent of the compound(s) being administered, as measured in an in vitro assay. Alternatively, an initial dosage for humans may be based upon dosages found to be effective in animal models of fibrotic diseases or conditions, such as the bleomycin-induced lung fibrosis mouse model. As one example, the initial dosage for each component of the pharmaceutical compositions outlined herein may be in the range of about 0.01 mg/kg/day to about 3000 mg/kg/day, or about 0.1 mg/kg/day to about 2000 mg/kg/day, or about 1 mg/kg/day to about 2000 mg/kg/day, or about 10 mg/kg/day to about 2000 mg/kg/day, or about 100 mg/kg/day to about 2000 mg/kg/day, or about 1000 mg/kg/day to about 2000 mg/kg/day can also be used. The dosages, however, may be varied depending upon the requirements of the patient, the severity of the condition being treated, and the compound(s) being employed. The size of the dose also will be determined by the existence, nature, and extent of any adverse side-effects that accompany the administration of a particular compound(s) in a particular patient. Determination of the proper dosage for a particular situation is within the skill of the practitioner. Generally, treatment is initiated with smaller dosages which are less than the optimum dose of the compound(s). Thereafter, the dosage is increased by small increments until the optimum effect under circumstances is reached. For convenience, the total daily dosage may be divided and administered in portions during the day, if desired.

The concentration of active compound in the drug composition will depend on absorption, distribution, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed composition. The active ingredient may be administered at once, or may be divided into a number of smaller doses to be administered at varying intervals of time.

Formulations suitable for oral administration can consist of (a) liquid solutions, such as an effective amount of the compound(s) suspended in diluents, such as water, saline or PEG 400; (b) capsules, sachets or tablets, each containing a predetermined amount of the active ingredient, as liquids, solids, granules or gelatin; (c) suspensions in an appropriate liquid; and (d) suitable emulsions. Tablet forms can include one or more of lactose, sucrose, mannitol, sorbitol, calcium phosphates, corn starch, potato starch, microcrystalline cellulose, gelatin, colloidal silicon dioxide, talc, magnesium stearate, stearic acid, and other excipients, colorants, fillers, binders, diluents, buffering agents, moistening agents, preservatives, flavoring agents, dyes, disintegrating agents, and pharmaceutically compatible carriers. Lozenge forms can comprise the active ingredient in a flavor, e.g., sucrose, as well as pastilles comprising the active ingredient in an inert base, such as gelatin and glycerin or sucrose and acacia emulsions, gels, and the like containing, in addition to the active ingredient, carriers known in the art.

Oral compositions will generally include an inert diluent or an edible carrier. They may be enclosed in gelatin capsules or compressed into tablets. For the purpose of oral therapeutic administration, the active compound can be incorporated with excipients and used in the form of tablets, troches, or capsules. Pharmaceutically compatible binding agents, and/or adjuvant materials can be included as part of the composition.

The active compound or pharmaceutically acceptable salt thereof can be administered as a component of an elixir, suspension, syrup, wafer, chewing gum or the like. Syrup may contain, in addition to the active compounds, sucrose as a sweetening agent and certain preservatives, dyes and colorings and flavors.

The active compound or pharmaceutically acceptable salts thereof can also be mixed with other active materials that do not impair the desired action, or with materials that supplement the desired action.

As used herein, the term pharmaceutically acceptable salt(s) refers to salts that retain the desired biological activity of the above-identified compounds and exhibit minimal or no undesired toxicological effects. Examples of such salts include, but are not limited to acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalacturonic acid. The compounds can also be administered as pharmaceutically acceptable quaternary salts known by those skilled in the art, which specifically include the quaternary ammonium salt of the formula —NR+Z—, wherein R is hydrogen, alkyl, or benzyl, and Z is a counter-ion, including chloride, bromide, iodide, —O-alkyl, toluenesulfonate, methylsulfonate, sulfonate, phosphate, or carboxylate (such as benzoate, succinate, acetate, glycolate, maleate, malate, citrate, tartrate, ascorbate, benzoate, cinnamoate, mandeloate, benzyloate, and diphenylacetate).

The compound(s) of choice, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Suitable formulations for rectal administration include, for example, suppositories, which consist of the packaged nucleic acid with a suppository base. Suitable suppository bases include natural or synthetic triglycerides or paraffin hydrocarbons. In addition, it is also possible to use gelatin rectal capsules which consist of a combination of the compound(s) of choice with a base, including, for example, liquid triglycerides, polyethylene glycols, and paraffin hydrocarbons.

Formulations suitable for parenteral administration, such as, for example, by intra-articular (in the joints), intravenous, intramuscular, intradermal, intraperitoneal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. In the practice of this invention, compositions can be administered, for example, by intravenous infusion, orally, topically, intraperitoneally, intravesically or intrathecally. Parenteral administration, oral administration, subcutaneous administration and intravenous administration are the preferred methods of administration. A specific example of a suitable solution formulation may comprise from about 0.1-100 mg/ml compound(s) and about 1000 mg/ml propylene glycol in water. Another specific example of a suitable solution formulation may comprise from about 0.1 or about 0.2 to about 100 mg/ml compound(s) and from about 800-1000 mg/ml polyethylene glycol 400 (PEG 400) in water.

A specific example of a suitable suspension formulation may include from about 0.2-30 mg/ml compound(s) and one or more excipients selected from the group consisting of: about 200 mg/ml ethanol, about 1000 mg/ml vegetable oil (e.g., corn oil), about 600-1000 mg/ml fruit juice (e.g., grape juice), about 400-800 mg/ml milk, about 0.1 mg/ml carboxymethylcellulose (or microcrystalline cellulose), about 0.5 mg/ml benzyl alcohol (or a combination of benzyl alcohol and benzalkonium chloride) and about 40-50 mM buffer, pH 7 (e.g., phosphate buffer, acetate buffer or citrate buffer or, alternatively 5% dextrose may be used in place of the buffer) in water.

A specific example of a suitable liposome suspension formulation may comprise from about 0.5-30 mg/ml compound(s), about 100-200 mg/ml lecithin (or other phospholipid or mixture of phospholipids) and optionally about 5 mg/ml cholesterol in water. For subcutaneous administration of a compound(s), a liposome suspension formulation including 5 mg/ml compound(s) in water with 100 mg/ml lecithin and 5 mg/ml compound(s) in water with 100 mg/ml lecithin and 5 mg/ml cholesterol provides good results.

The formulations of compound(s) can be presented in unit-dose or multi-dose sealed containers, such as ampoules and vials. Injection solutions and suspensions can be prepared from sterile powders, granules, and tablets of the kind previously described.

The pharmaceutical preparation is preferably in unit dosage form. In such form the preparation is subdivided into unit doses containing appropriate quantities of the compound(s). The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form. The composition can, if desired, also contain other compatible therapeutic agents, discussed in more detail, below.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation (CA) and Gilford Pharmaceuticals (Baltimore, Md.). Liposomel suspensions may also be pharmaceutically acceptable carriers. These may be prepared according to methods known to those skilled in the art, for example, as described in U.S. Pat. No. 4,522,811 (which is incorporated herein by reference in its entirety). For example, liposome formulations may be prepared by dissolving appropriate lipid(s) (such as stearoyl phosphatidyl ethanolamine, stearoyl phosphatidylcholine, arachadoyl phosphatidylcholine, and cholesterol) in an inorganic solvent that is then evaporated, leaving behind a thin film of dried lipid on the surface of the container. Aqueous solutions of the active compound or its monophosphate, diphosphate, and/or triphosphate derivatives are then introduced into the container. The container is then swirled by hand to free lipid material from the sides of the container and to disperse lipid aggregates, thereby forming the liposomal suspension.

Accordingly, the compositions described herein find use in the treatment of fibrotic conditions. The repair of damaged tissue is a fundamental biological process that is necessary for survival. However, in cases of repeated or sustained injury, the repair process may become abnormal and ECM proteins may build up to a pathological level. In the experiments described herein, tissue damage was caused by diverse agents: bleomycin in the lung and ANIT in the liver. These models have in common the fact that the irritant dramatically increased the synthesis of tissue collagen above the levels obtained in non-treated controls. This effect was reflected in an increase in the total collagen pool and the absolute amount of new collagen synthesized in the tissues. In both models, noscapine dose-dependently blocked the increase in new collagen synthesis, both fractional and absolute, as well as the total amount of collagen present in the tissues.

Noscapine has 2 main pharmacological activities that are of interest in these experiments. Noscapine is a MT modulating agent (Ye et al. 1998) as well as a bradykinin antagonist (Mahmoudian et al. 2001). Without being bound by theory, this combination of activity may make noscapine an especially interesting novel agent for the treatment of fibrosis.

During the normal wound healing process, damaged epithelial and/or endothelial cells release mediators to activate fibroblasts. As they migrate into the wound, fibroblasts transform into alpha-SMA expressing myofibroblasts that play a crucial role the pathological tissue remodeling (review Hinz et al. 2007). The dynamic properties of MT allow the reorientation of the MT network when large cells undergo migration. Without being bound by theory, it is thought that because noscapine decreases the dynamicity of MT, it could decrease the movement of cells such as fibroblasts into the wounded area. Liao (et al 1995) found in vitro that a low concentration of substances, such as nocodazole, that interfere with MT dynamics, but not the actual level of MT in the cell, decrease the rate of fibroblast locomotion into a wounded area of a fibroblast culture. Anti-fibrotic effects of MT modulating agents such as noscapine had not previously been demonstrated in vivo, however.

MT may also play an important role in the negative regulation of the TGF-β/Smad signaling pathway. In vivo, Liu (et al, 2005) found that low concentrations of a MT interfering agent (paclitaxel) significantly dampened TGF-β signaling in the nude mouse model of scleroderma. Paclitaxel markedly suppressed Smad2 and Smad3 phosphorylation and decreased collagen deposition in the SSc grafts (Liu et al, 2005). The TGF-β Smad signaling pathway has been linked causally to the induction of fibrosis (reviews: Wells, 2000, Verrecchia and Mauviel, 2007). TGF-β is chemotactic for fibroblasts, for example, and induces fibroblasts to synthesize ECM while also increasing the production of protease inhibitors that prevent the enzymatic breakdown of the ECM; TGF-β regulates lymphocyte function and increases endothelial cell apoptosis while inhibiting smooth muscle cell apoptosis.

At low concentrations in vitro, all MT interfering agents disrupt MT dynamics, but these agents act on microtubules at different binding sites which causes them to have distinctive effects (Jordan, 2002). MT interfering agents fall into 2 general classes: compounds such as colchicine, nocodazole and vinca alkaloids that inhibit MT polymerization and compounds such as the taxoids (e.g. paclitaxel) that promote MT polymerization (Jordan, 2002). Colchicine, an anti-inflammatory drug that has been used in the treatment of gout, binds tublins and results in a disruption of MT polymerization. Colchicine has been tested as an alternative to corticosteroid and/or immunosuppressive drugs, in the treatment of IPF (Douglas et al 1998) and was found to be safer, but not effective (i.e., no more effective than conventional therapy, which was ineffective). Although noscapine is chemically similar to colchicine, it binds to different site on tubulin (Ye et al, 1998). Upon binding, noscapine induces a conformational change in tubulin that promotes polymerization and assembly of MT rather than disrupting polymerization, and may act in a general manner to slow the MT disassembly/re-assembly cycle.

Many MT interfering agents, such as the toxoids, have been exploited as anti-cancer therapies because of their ability to limit cell proliferation. However the side effect profile of these agents would make them unsuitable for the treatment of fibrosis. In fact disruption of MT network by some of these agents leads to ligand-independent Smad nuclear accumulation with transcription of TGF-β-responsive genes and increases TGF-β-induced Smad activity, all activities that would be more likely to be fibrogenic in nature. Paclitaxel treatment had been linked to the development of a scleroderma-like syndrome in some patients (e.g. De Angelis et al, 2003). Whether noscapine would exert pro-fibrotic, anti-fibrotic or neutral actions in vivo in animal models of fibrotic disease or condition was therefore not predictable.

In addition to its MT modulating effects, noscapine also behaves as a non-competitive bradykinin antagonist (Mahmoudian et al. 2001), a mechanism that is responsible for at least some of its cough suppressant activity (Ebrahimi et al, 2003). Bradykinin elicits a variety of biological effects: hypotension, bronchoconstriction, gut and uterine contraction, epithelial secretion in airway, gut, and exocrine glands, vascular permeability, pain, connective tissue proliferation, cytokine release, and eicosanoid formation (Steranka et al, 1989). Bradykinin may be implicated in the induction of fibrosis, although evidence to the contrary exists (e.g. Sancho-Bru et al, 2007; Helske et al, 2007). By inducing fibroblast proliferation, the transition of lung fibroblasts into myofibroblasts, and promoting collagen production, bradykinin may be involved in bronchial remodeling and lung fibrosis (Vancheri et al, 2005). Bradykinin also increased collagen mRNA, secretion of TIMP and TGF-β in vascular smooth muscle cells (Douillet et al, 2000). Treatment with bradykinin antagonists was able to block the fibrogenic effects of bradykinin in a model of myocardial remodeling (Koike et al, 2005). Therefore, it is possible that some of the anti-fibrotic action of noscapine in the bleomycin and ANIT models was due to bradykinin antagonist activity in addition to its MT modulating activity.

In summary, noscapine appears to be a novel antifibrotic drug that has the potential to treat a variety of fibrotic conditions. The antifibrotic activity of noscapine may be attributed to its unique pharmacological profile as a MT modulating agent and/or as a bradykinin antagonist.

The following non-limiting examples further illustrate the invention disclosed herein.

### EXAMPLES

### Example I

### Bleomycin-Induced Lung Fibrosis

Idiopathic pulmonary fibrosis (IPF) is a chronic and often fatal disorder characterized by excessive deposition of ECM resulting in extensive tissue remodeling that impairs lung function. Conventional treatments, such as those involving immunosuppressants, are ineffective in controlling or preventing disease progression.

Endotracheal administration of bleomycin to rodents has become the standard experimental model of human interstitial lung fibrosis. Bleomycin toxicity (review Sleijfer, 2001) occurs mainly in the lungs and skin due to a lack of the inactivating enzyme, bleomycin hydrolase, in these tissues. Bleomycin induces cytotoxicity through the induction of free radicals which then cause breaks in DNA. Many of the histological alterations of IPF are reproduced by the administration of bleomycin (reviewed in Grande et al. 1998): marked distortion of the alveoli, capillary remodeling, and excessive deposits of ECM, especially collagen. In the bleomycin model, as well as in IPF, TGF-β is a major molecular mediator of fibroblast proliferation and increased collagen synthesis.

Bleomycin administration induces at least a three fold increase in the fractional synthesis of OHP as measured by GC/MS. Inhibition or reversal of the fibrotic response is determined by a reduction in OHP synthesis when compared with the bleomycin-vehicle control.

Method:

Animals: Female C57BU6 mice, 10 weeks of age at the start of the study were used. Food and water were available ad lib for the study duration. Study groups are as stated in table 1.

TABLE 1
summary of experimental groups in bleomycin-induced fibrosis

| Dose | | |
|---|---|---|
| noscapine | Route | n/group |
| 300 mg/kg | po | 10 |
| 300 mg/kg | Diet | 5 |
| | 2 g/kg food | |
| 10 mg/kg | ip | 4 |
| 30 mg/kg | | |
| 100 mg/kg | | |

Protocol: Bleomycin (Sigma-Aldrich) was dissolved in sterile saline at a concentration of 1.05 U/ml. 30 μl of the bleomycin solution or the same volume of vehicle (sham control) was installed into the trachea of female C57BI/6 mice by the trans-oral route to yield a dose of 1.5 U/kg of bleomycin. On the same day, the mice were labeled with 8% ²H₂O and administration of noscapine was begun. Label (8% ²H₂O in drinking water) and drug administration continued for the 2 week study duration. The doses of noscapine and route of administration are as stated in table 1 (po signifies oral gavage; ip, intra-peritoneal).

On the day following the last drug administration, the mice were placed under heavy anesthesia and blood was collected via cardiac puncture. The lung was perfused with isotonic saline, removed and weighed. The lung was homogenized with a MiniBeadbeater-96™ (Biospec, Bartlesville, Okla.) bead mill, and the total volume of homogenate was determined. The homogenate was subjected to acetone precipitation in order to obtain the total tissue protein for OHP assessment. The proteins were hydrolyzed by incubation in HCl, dried under vacuum and then suspended in a solution of 50% acetonitrile, 50 mM K₂HPO₄ and pentafluorobenzyl bromide before incubation. Derivatives were extracted into ethyl acetate, and the top layer was removed and dried by vacuum centrifugation. In order to acetylate the hydroxyl moiety of hydroxyproline, samples were incubated with a solution of acetonitrile, N-Methyl-N-[tert-butyldimethyl-silyl]trifluoroacetamide (MTBSTFA) and methylimidizole. This material was extracted in petroleum ether and dried with Na₂SO₄. The derivatized OHP was analyzed by GC/MS, performed in the negative chemical ionization mode. Selected ion monitoring was performed on ions with mass-to-charge ratios (m/z) 445, 446, and 447, which include all of the carbon-hydrogen bonds from OHP. Incorporation of ²H into OHP was calculated as the molar fraction of molecules with one excess mass unit above the natural abundance fraction (EM1). Fractional synthesis (f) of collagen was calculated as the ratio of the EM1 value in protein-bound OHP to the maximal value possible at the body ²H₂O enrichment present.

In addition to the GC/MS analysis, the absolute amount of collagen in the samples was determined by a chloramine-t assay of total OHP, a well established colorimetric method for determining OHP content. Samples of the original homogenate (400 μl) were hydrolyzed (HCl, 500 μl), dried, re-suspended in 1 ml of assay buffer, and incubated overnight. 150 μl of the suspension was pipetted into wells of a 96 well plate. 75 μl of chloramine-t was added to the well and the plate was incubated at room temperature for 20 min. Dimethylaminobenzaldehyde (75 μl) was then added to each well. The plate was incubated at 60° C. for 15 min before being read on a microquant (Bio-Tek Instruments, Inc. MQX200) at 540 nm. OHP standards were prepared and analyzed with the samples. Results were analyzed using GraphPad Prism to interpolate values from the standard curve.

The differentiation of fibroblastic cells into myofibroblasts which express α-smooth muscle actin (α-SMA) is a major process the development of fibrosis. αSMA is a well recognized indicator of activated myofibroblasts. A small sample (5 to 20 mg) of the lung was excised from the center of the left lobe, immediately fixed in formalin, and sent for histological examination (Premier Laboratory LLC, Longmont Colo., USA). Briefly, paraffin embedded slides were prepared from the formalin-fixed tissue and then stained using an antibody for αSMA. Prepared slides were scanned, photographed, and subsequently analyzed for the presence of αSMA positive cells using ImageScope software (Aperio Technologies Inc., Vista Ca).

Statistics: An analysis of variance followed by a Dunnett's test for comparison with drug vehicle/bleomycin control was used to analyze the data (SigmaStat). Data were considered significant at p<0.05.

Results:

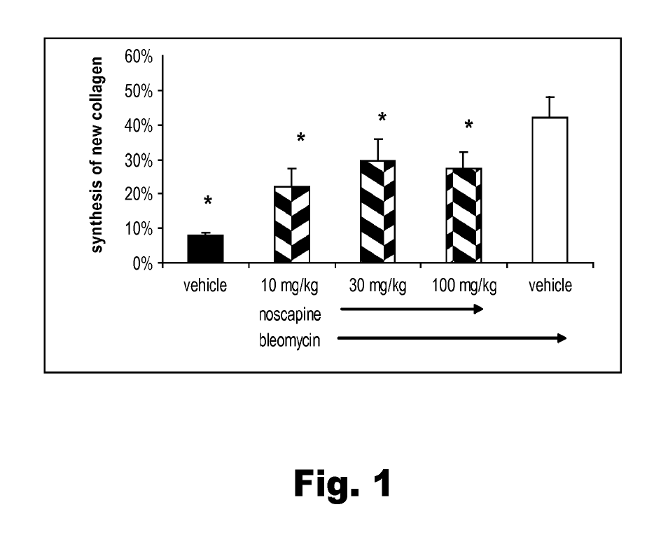

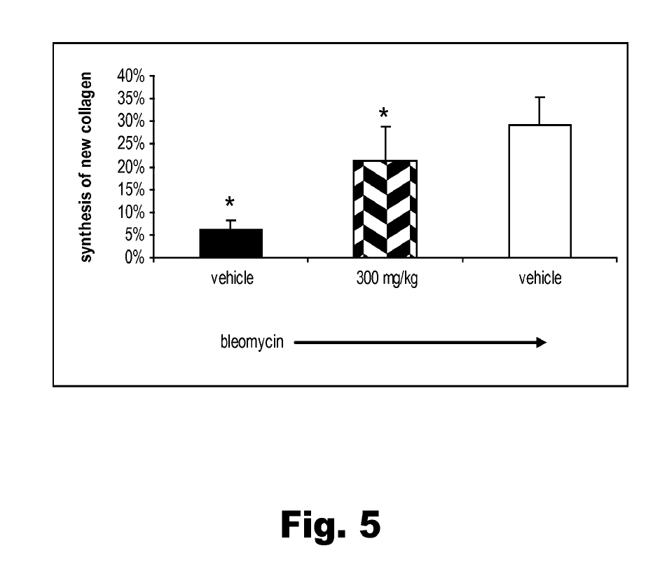

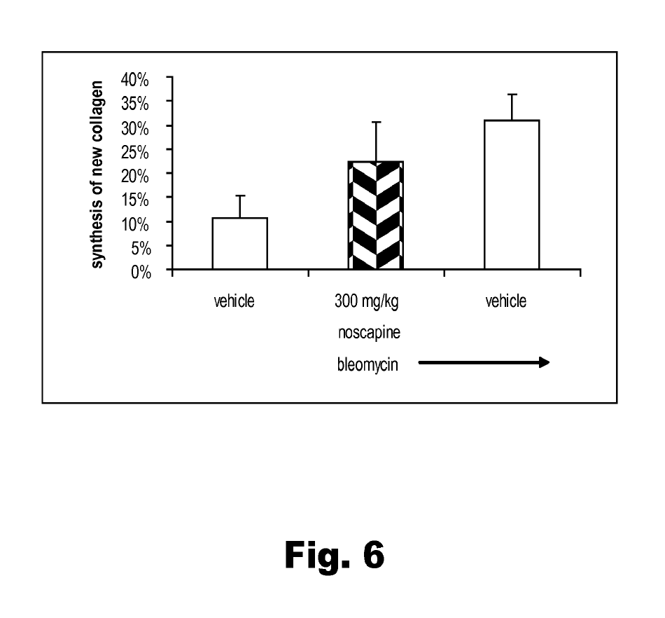

Bleomycin induced a significant increase in the fractional synthesis of collagen (FIGS. 1, 5 and 6). Noscapine blocked the bleomycin-induced increase of collagen synthesis whether injected intraperitoneally (ip) (FIG. 1) or administered by oral gavage (po) (FIG. 5). Noscapine also tended to reduce the percent of newly synthesized collagen when administered in diet (FIG. 6).

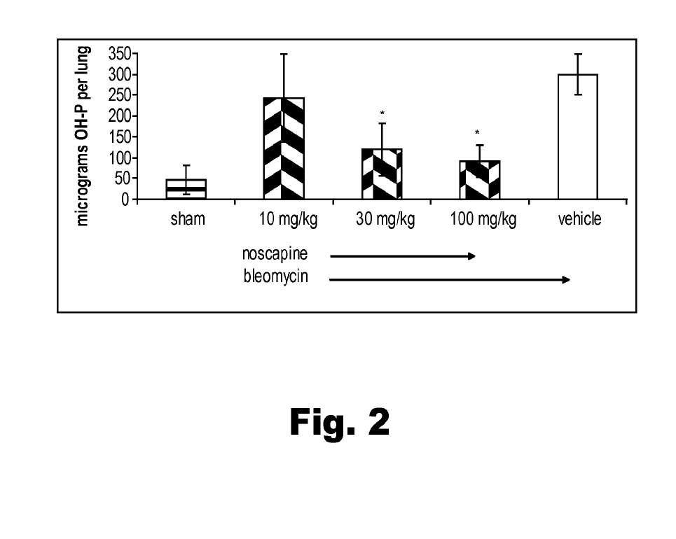

Bleomycin also increased the total collagen (OHP) content of the lung as assessed by the chloramine-t assay. This effect was blocked by noscapine in a dose related manner (FIG. 2).

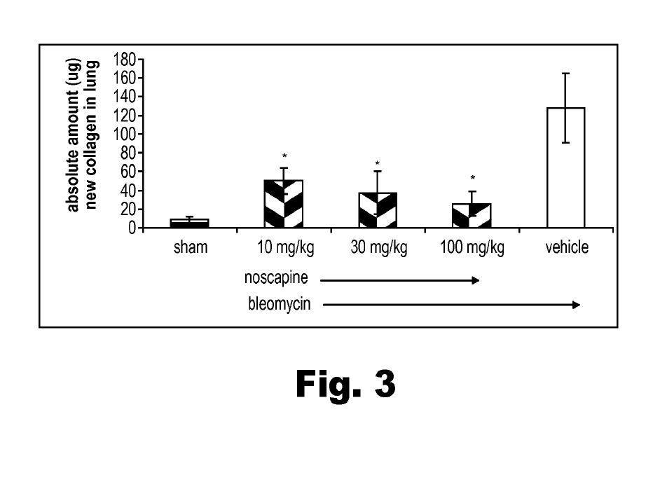

The absolute amount of newly synthesized collagen (calculated as fractional synthesis (f) times the total OHP content) in the lung was significantly decreased by noscapine (FIG. 3).

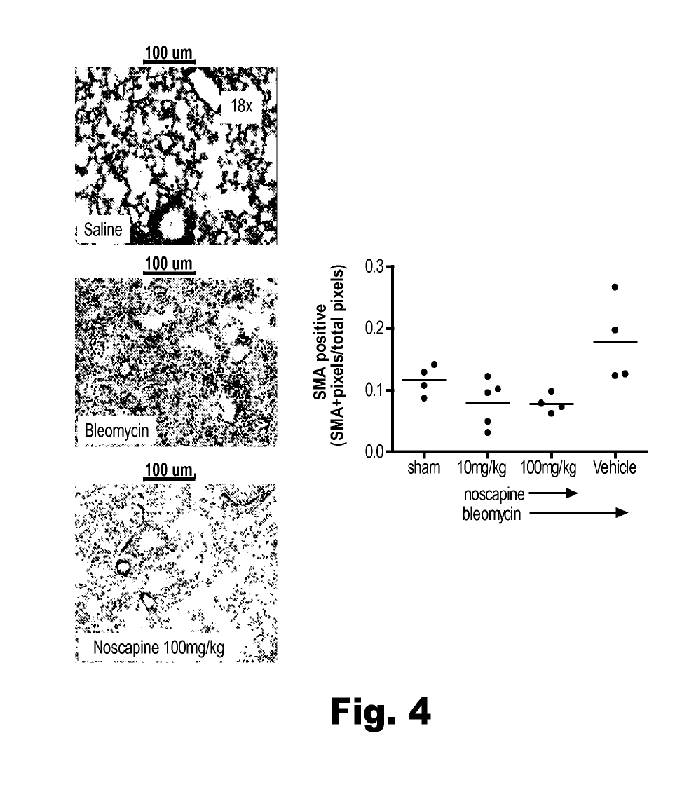

Noscapine significantly decreased the number of alpha smooth muscle actin (αSMA) positive cells (FIG. 4) relative to the vehicle+bleomycin control group.

### Example II

### ANIT-Induced Liver Fibrosis

Alpha-Naphthylisothiocyanate (ANIT) is a hepatotoxicant that damages biliary cells and hepatocytes. Prolonged exposure to ANIT induces bile duct hyperplasia and biliary fibrosis, as well as liver injury. ANIT is conjugated with glutathione in hepatocytes and secreted into the bile (reviewed in Xu, 2004). However, the ANIT-glutathione complexes are unstable and rapidly dissociate in the bile causing the hepatocytes to reuptake the drug. Recycling can continue for many rounds, delaying ANIT elimination and depleting glutathione. In addition to its direct cytotoxic effects, ANIT also induces a hepatic inflammatory response that contributes to the tissue injury. Chronic administration of ANIT to rodents results in a significant increase in liver collagen and is used as an animal model of liver fibrosis (Xu et al, 2004). Using KineMed's heavy water labeling and GC/MS technology, ANIT administration has been found to induce at least a three fold increase in the fractional synthesis of collagen. Inhibition or reversal of the fibrotic response is determined by a reduction in collagen synthesis when compared with the ANIT-vehicle control.

Method:

Subjects: Female C57BU6 mice, 10 weeks of age at the start of the study were used. The animals were housed in groups of 5, under standard laboratory conditions, with food and water available ad lib. Study groups are as stated in table 2 (po signifies oral gavage).

TABLE 2
summary of experimental groups for ANIT-induced liver fibrosis.

| Dose | | | |
|---|---|---|---|
| noscapine | | Route | n/group |
| ~300 | mg/kg | Diet | 5 |
| | | 2 g/kg food | |
| 300 | mg/kg | po | 5 |
| 10 | mg/kg | po | 5 |
| 30 | mg/kg | | 5 |
| 100 | mg/kg | | 4 |

Protocol:

Mice were fed a diet adulterated with 0.05% (w/w) ANIT or a control diet for two weeks in order to induce a fibrotic response in the liver. The mice were labeled with 8% ²H₂O (Spectra Stable Isotopes, Columbia, Md.) beginning on the day that ANIT administration began. On the same day, administration of noscapine HCl (Sigma-Aldrich) was begun as listed in table 2. ANIT, label and noscapine administration continued for 2 weeks.

On the day following the last drug administration, mice were placed under heavy anesthesia, and the liver was perfused and removed. Liver tissue was homogenized with a MiniBeadbeater 96™ bead mill (Biospec, Bartlesville, Okla.) followed by acetone precipitation and vacuum drying. Proteins were hydrolyzed in 6 N HCl (110° C., 16 hours), dried under vacuum and suspended in 1 mL 50% acetonitrile, 50 mM K2HPO4, pH 11. Pentafluorobenzyl bromide was added before the sealed mixture was incubated (100° C.; 1 hour) and extracted into ethyl acetate. The top layer was removed and dried by addition of solid Na2SO4 followed by vacuum centrifugation. Samples were incubated with 50 μL methyl imidazole and 100 μL MTBSTFA, (100° C. for 30 min). The derivative was extracted in water/petroleum ether and dried with Na2SO4. Selected ion monitoring was at mass-to-charge ratios of 445, 446, and 447 for the OH-proline derivatives. Mole % excess M1 enrichment (EM1) and fractional synthesis (f) are calculated.

In addition to the GC/MS analysis, the absolute amount of OHP in the samples was determined by a chloramine-t assay as described above for the bleomycin studies.

The effect of noscapine on the ANIT-induced changes in collagen content was confirmed histologically. A small sample (75 to 80 mg) of the liver was excised from the lateral right lobe, immediately fixed in formalin, and sent for histological examination (Premier Laboratory LLC, Longmont Colo., USA). Briefly, paraffin embedded slides were prepared from the formalin-fixed tissue and then treated with Masson's Trichrome stain for collagen distribution. Prepared slides were scanned, photographed, and subsequently analyzed for collagen content using ImageScope software (Aperio Technologies Inc., Vista Ca).

Statistics: An analysis of variance followed by a Dunnett's test for comparison with ANIT/vehicle control was used to analyze the data (SigmaStat). Data were considered significant at p<0.05.

Results:

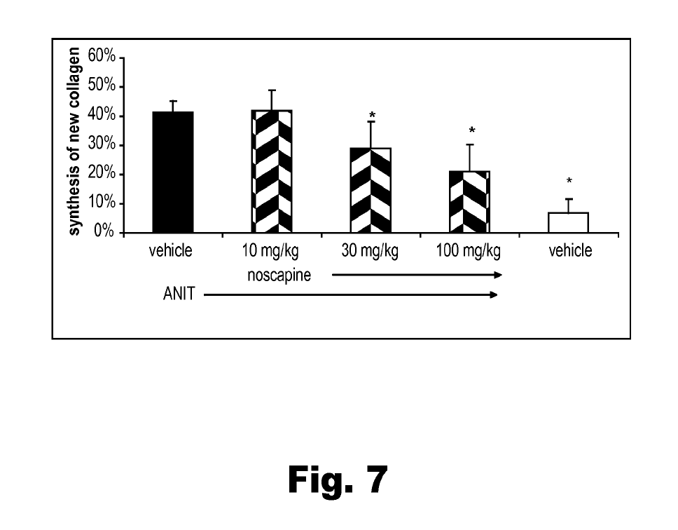

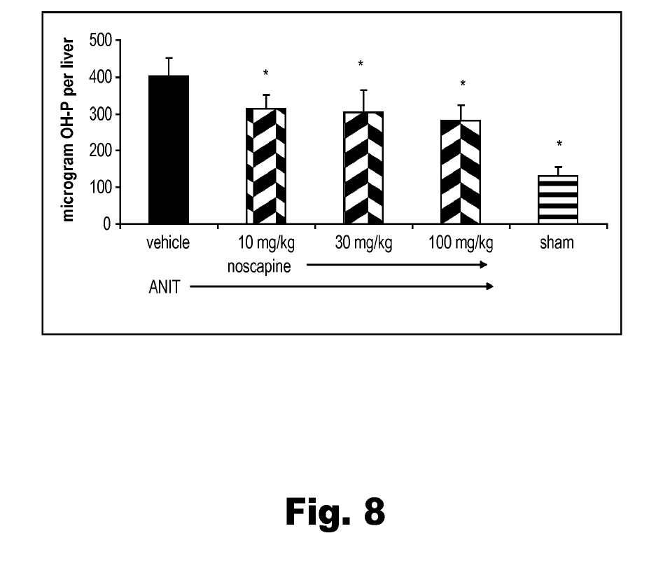

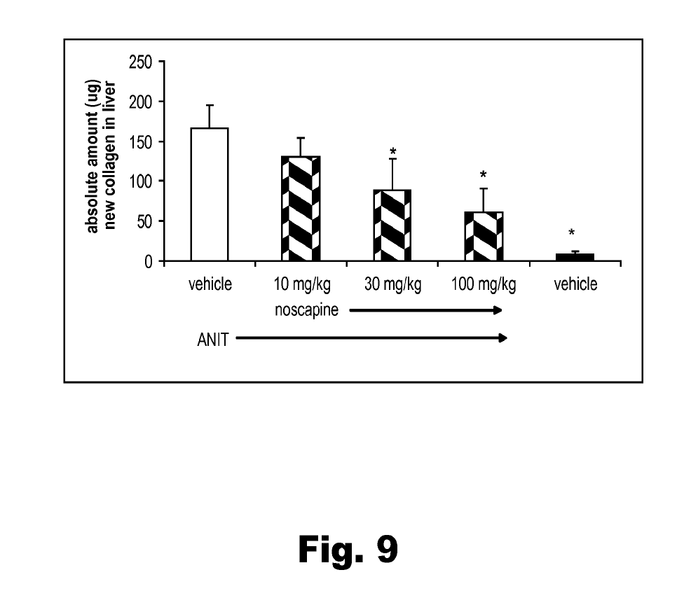

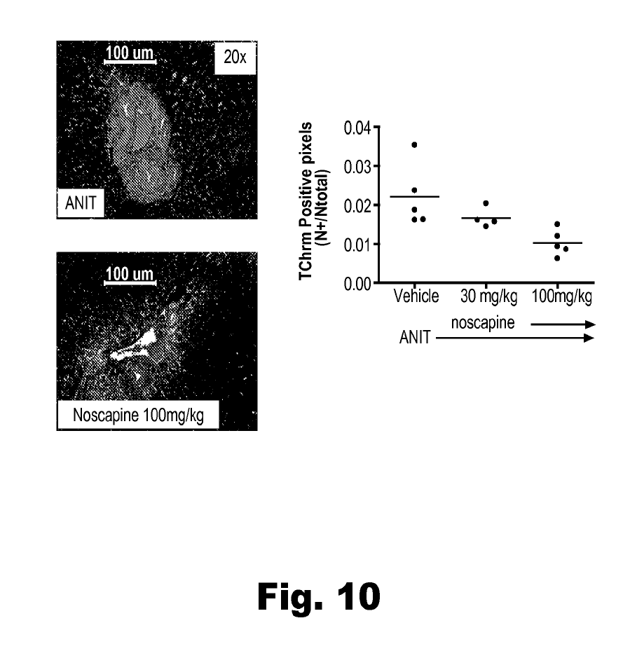

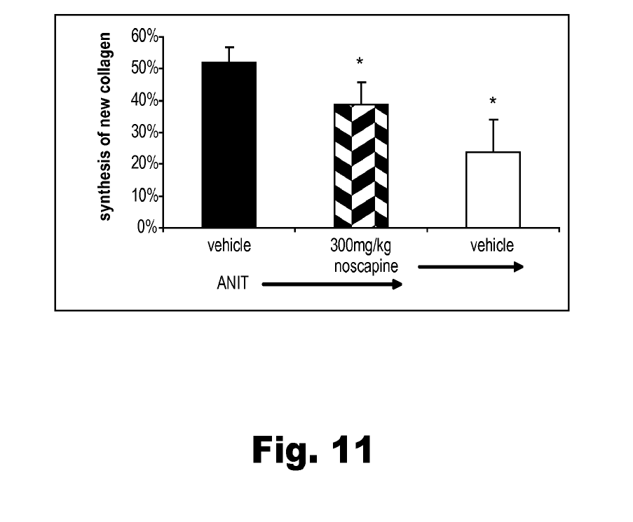

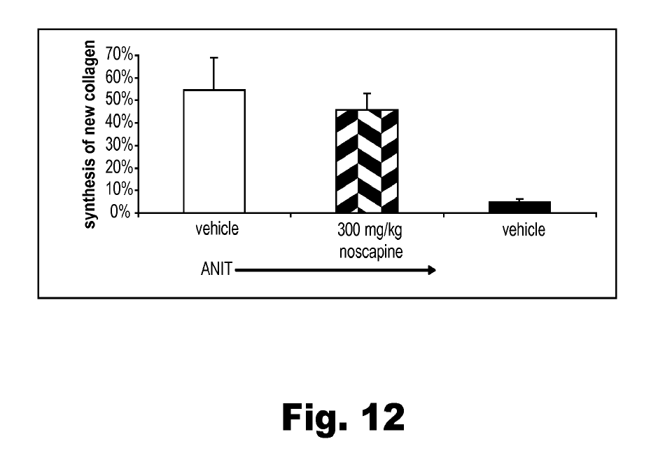

Administration of 0.05% ANIT in diet over a two week period resulted in a significant increase in the percentage of newly synthesized hydroxyproline (OHP) in the liver (FIGS. 7, 11 and 12). The absolute amount of new and total OHP (as measured by the chloramine-t test) in the liver were also significantly increased by administering ANIT (FIGS. 8 and 9). When administered by oral gavage (po), noscapine dose dependently reduced the percent of newly synthesized collagen (FIG. 7). The total amount of OHP and the amount of newly synthesized collagen in the whole liver were also significantly reduced when compared to the ANIT plus vehicle control group (FIGS. 8 and 9). A significant decrease in collagen content when noscapine was administered with ANIT was confirmed histologically (FIG. 10). In separate studies, a 300 mg/kg dose of noscapine administered either in the diet (FIG. 11) or by oral gavage (FIG. 12) also tended to decrease the amount of newly synthesized collagen.

Although the foregoing invention has been described in some detail by way of illustration and examples for purposes of clarity of understanding, it will be apparent to those skilled in the art that certain changes and modifications may be practiced without departing from the spirit and scope of the invention. Therefore, the description should not be construed as limiting the scope of the invention, which is delineated by the appended claims.

All publications, patents, and patent applications cited herein are hereby incorporated by reference in their entirety for all purposes to the same extent as if each individual publication, patent, or patent application were specifically and individually indicated to be so incorporated by reference.

### CITED REFERENCES

De Angelis R, Bugatti L, Cerioni A, Del Medico P, Filosa G. Diffuse scleroderma occurring after the use of paclitaxel for ovarian cancer. Clin Rheumatol. 2003, 22:49-52.
Dong C, Li Z, Alvarez R Jr, Feng X H, Goldschmidt-Clermont P J (2000)
Microtubule binding to Smads may regulate TGF beta activity. Mol Cell 5: 27-34.
Douillet C D, Velarde V, Christopher J T, Mayfield R K, Trojanowska M E, Jaffa A A. Mechanisms by which bradykinin promotes fibrosis in vascular smooth muscle cells: role of TGF-beta and MAPK. Am J Physiol Heart Circ Physiol. 2000 December; 279(6):H2829-37.
Ebrahimi S A, Zareie M R, Rostami P, Mahmoudian M. Interaction of noscapine with the bradykinin mediation of the cough response. Acta Physiol Hung. 2003; 90(2):147-155
Empey D W, Laitinen L A, Young G A, Bye C E, Hughes D T.
Comparison of the antitussive effects of codeine phosphate 20 mg, dextromethorphan 30 mg and noscapine 30 mg using citric acid-induced cough in normal subjects. Eur J Clin Pharmacol. 1979; 16(6):393-397
Gardner J L, Turner S M, Bautista A, Lindwall G, Awada M, Hellerstein M K.
Measurement of liver collagen synthesis by heavy water labeling: effects of profibrotic toxicants and antifibrotic interventions.
Am J Physiol Gastrointest Liver Physiol. 2007 June; 292(6):G1695-1705.
Grande N R, Peao M N, deSa C M, Aguas A P. Lung fibrosis induced by bleomycin: structural changes and overview of recent advances. Scanning Microscopy, 1998 12, 487-494.
Hellerstein M K. Exploiting Complexity and the Robustness of Network Architecture for Drug Discovery. J Pharmacol Exp Ther. 2008 April; 325(1):1-9.
Helske S, Laine M, Kupari M, Lommi J, Turto H, Nurmi L, Tikkanen I, Werkkala K, Lindstedt K A, Kovanen P T. Increased expression of profibrotic neutral endopeptidase and bradykinin type 1 receptors in stenotic aortic valves. Eur Heart J. 2007 August; 28(15):1894-1903
Hinz B, Phan S H, Thannickal V J, Galli A, Bochaton-Piallat M L, Gabbiani G.
The myofibroblast: one function, multiple origins. Am J. Pathol. 2007 June; 170(6):1807-16.
Jordan M A. Mechanism of action of antitumor drugs that interact with microtubules and tubulin. Curr Med Chem Anticancer Agents. 2002 January; 2(1):1-17.
Ke Y, Ye K, Grossniklaus H E, Archer D R, Joshi H C, Kapp J A.
Noscapine inhibits tumor growth with little toxicity to normal tissues or inhibition of immune responses. Cancer Immunol Immunother. 2000 July; 49(4-5):217-225.
Koike M K, de Carvalho Frimm C, de Lourdes Higuchi M. Bradykinin B2 receptor antagonism attenuates inflammation, mast cell infiltration and fibrosis in remote myocardium after infarction in rats. Clin Exp Pharmacol Physiol. 2005 December; 32(12):1131-1136
Liu X, Zhu S, Wang T, Hummers L, Wigley F M, et al. Paclitaxel modulates TGFβ signaling in scleroderma skin grafts in immunodeficient mice. 2005 PLoS Med 2: e354.
Mahmoudian M, Mojaverian N. Effect of noscapine, the antitussive opioid alkaloid, on bradykinin-induced smooth muscle contraction in the isolated ileum of the guinea-pig Acta Physiol Hung. 2001; 88(3-4):231-237
Mahmoudian M, Mehrpour M, Benaissa F, Siadatpour Z. A preliminary report on the application of noscapine in the treatment of stroke Eur J Clin Pharmacol. 2003 November; 59(8-9):579-581.
Sancho-Bru P, Bataller R, Fernandez-Varo G, Moreno M, Ramalho L N, Colmenero J, Mari M, Clària J, Jiménez W, Arroyo V, Brenner D A, Ginès P. Bradykinin attenuates hepatocellular damage and fibrosis in rats with chronic liver injury. Gastroenterology. 2007 December; 133(6):2019-2028
Sleijfer S. Bleomycin-induced pneumonitis. Chest. 2001 August; 120(2):617-624
Steranka L R, Farmer S G, Burch R M. Antagonists of B2 bradykinin receptors. FASEB J. 1989 July; 3(9):2019-2025
Turner S M, Hellerstein M K. Emerging applications of kinetic biomarkers in preclinical and clinical drug development. Curr Opin Drug Discov Devel. 2005 January; 8(1):115-26.
Vancheri C, Gili E, Failla M, Mastruzzo C, Salinaro E T, Lofurno D, Pistorio M P, La Rosa C, Caruso M, Crimi N. Bradykinin differentiates human lung fibroblasts to a myofibroblast phenotype via the B2 receptor. J Allergy Clin Immunol. 2005 December; 116(6):1242-1248
Verrecchia F, Mauviel A. Transforming growth factor-beta and fibrosis.
World J. Gastroenterol. 2007 Jun. 14; 13(22):3056-3062.
Wells R G. Fibrogenesis. V. TGF-beta signaling pathways.
Am J Physiol Gastrointest Liver Physiol. 2000 November; 279(5):G845-850.
Wynn T A. Common and unique mechanisms regulate fibrosis in various fibroproliferative diseases. The Journal of Clinical Investigations, 2007, 117, 524-529.
Xu J, Lee G, Wang H, Vierling J M, Maher J J. Limited role for CXC chemokines in the pathogenesis of alpha-naphthylisothiocyanate-induced liver injury.
Am J Physiol Gastrointest Liver Physiol. 2004 September; 287(3):G734-41
Ye K, Ke Y, Keshava N, Shanks J, Kapp J A, Tekmal R R, Petros J, Joshi H C.
Opium alkaloid noscapine is an antitumor agent that arrests metaphase and induces apoptosis in dividing cells. Proc Natl Acad Sci USA. 1998 Feb. 17; 95(4):1601-1606.
Zhou J, Panda D, Landen J W, Wilson L, Joshi H C.
Minor alteration of microtubule dynamics causes loss of tension across kinetochore pairs and activates the spindle checkpoint.
J Biol. Chem. 2002 May 10; 277(19):17200-8.

We claim:

1. A method of treating a fibrotic disease or condition in a patient comprising administering noscapine and a pharmaceutical carrier to said patient.

2. The method according to claim 1 wherein the onset of the fibrotic disease or condition is delayed.

3. The method according to claim 1 wherein the severity of the fibrotic disease or condition is reduced.

4. The method according to claim 1, 2 or 3 wherein following said administering, the level of extracellular matrix proteins is reduced relative to the levels prior to said administering.

5. The method of claim 4, wherein an extracellular matrix protein is collagen.

6. The method of claim 1, 2, 3, 4 or 5, wherein said noscapine is a noscapine analog.

7. A method of treating a fibrotic disease or condition in a patient comprising administering a microtubule modulating agent and a pharmaceutical carrier to said patient.

8. The method of claim 1, 2, 3, 4, 5, 6 or 7, wherein said method further comprises administering an agent selected from the group consisting of ACE inhibitors, anti-fibrotics and anti-inflammatory agents.

9. The method of any of the preceding claims, wherein said administration is by oral administration, intravenous administration, subcutaneous administration, intra-dermal, topical, rectal suppository, aerosolized, intra-articular and intra-muscular administration.
